(19) United States
(12) Patent Application Publication
(10) Pub. No.: US 2026/0308097 A1
(43) Pub. Date: Oct. 8, 2026

(54) HYDROFORMYLATION PROCESS

(71) Applicant: Dow Technology Investments LLC, Midland, MI (US)

(72) Inventors: Christophe R. Laroche, Bartlesville, OK (US); Glenn A. Miller, Hacker Valley, WV (US); Michael A. Brammer, Lake Jackson, TX (US); Jason F. Giles, Horse Shoe, NC (US); Amarnath Singh, Pearland, TX (US)

(21) Appl. No.: 19/481,460

(22) PCT Filed: May 17, 2024
(86) PCT No.: PCT/US2024/029841

§ 371 Date: Nov. 4, 2025

Related U.S. Application Data
(60) Provisional application No. 63/517,505, filed on Aug. 3, 2023.

(51) Int. Cl. C07C 45/50

(52) U.S. Cl. CPC C07C 45/505

(57) ABSTRACT

The process of the invention is such a continuous hydroformylation process comprising: (a) removing from a reactor a crude product; (b) sending the crude product to a vaporizer; (c) separating the crude product in the vaporizer to produce a catalyst-containing liquid stream and a gas phase stream; and (d) maintaining an average CO partial pressure in the vaporizer of less than 15 psia (103 kPa) with reduced H₂ content.

### BACKGROUND OF THE INVENTION

The invention relates to a hydroformylation process. More specifically it relates to such a process wherein the amount of heavies in a catalyst recycle stream is controlled. More specifically it relates to such a process wherein the loss of catalyst stability is minimized.

It is well known that aldehydes can be produced by reacting olefins with carbon monoxide and hydrogen in the presence of a metal-organophosphorus ligand complex catalyst, and that preferred processes involve continuous hydroformylation and recycling of a catalyst solution containing a metal-organophosphorus ligand complex catalyst wherein the metal is selected from Groups 8, 9, or 10. Rhodium is a preferred Group 9 metal. U.S. Pat. Nos. 4,148,830, 4,717,775, and 4,769,498 disclose examples of this process. The resulting aldehydes can be used to produce a host of products including alcohols, amines, and acids. It is common practice to employ a vaporizer following the reaction zone for the purpose of separating products from the catalyst.

It is known that hydroformylation catalysts comprising rhodium and organophosphite ligands are capable of very high reaction rates; see, “Rhodium Catalyzed Hydroformylation,” van Leeuwen, Claver, Kluwer Academic Pub. (2000). Such catalysts have industrial utility, as they can be used to increase production rates, or to efficiently hydroformylate internal and/or branched internal olefins, which react more slowly than linear alpha olefins. However, it is also known, e.g., from U.S. Pat. No. 4,774,361, that under some conditions these catalysts lose rhodium in liquid recycle hydroformylation processes. A continuous loss of rhodium can increase catalyst costs dramatically, as rhodium is prohibitively expensive.

Although the exact cause of rhodium loss is unclear, it has been hypothesized in U.S. Pat. No. 4,774,361 and elsewhere that the loss is exacerbated by the low concentration of carbon monoxide (CO) and high temperature environment of a typical product separation step. U.S. Pat. No. 6,500,991 describes a means of slowing the loss of rhodium in an organophosphite-promoted process by cooling the concentrated catalyst following product removal, and then adding CO to the concentrated stream. U.S. Pat. No. 6,500,991 also describes adding CO to a depressurization/flash vessel prior to the separation step. For either option, the total pressure in the separation zone is taught to be less than or equal to 1 bar. Thus, the process of U.S. Pat. No. 6,500,991 attempts to stabilize the catalyst before and after the separation zone without directly addressing losses that may occur during the harsh environment of the separation step.

U.S. Pat. No. 8,404,903 describes a means of removing aldehyde product at greater than atmospheric pressure while employing relatively moderate temperatures. However, that process offers no means to control the CO content beyond changing the condenser temperature of the separation zone. This means of control is limited to a narrow range of CO partial pressures and requires an expensive refrigeration unit to condition such a large flow of gases. At the maximum total pressure (100 psia) and mole percent CO (16%) described in U.S. Pat. No. 8,404,903, a maximum CO partial pressure of 16 psia is possible, although at this high pressure, the separation zone production rate is unacceptably low, even for removal of the relatively volatile C₅ aldehyde. This is due to the fact that an acceptable balance of vaporizer temperature, recycle gas flow, and total pressure are required to achieve an acceptable product recovery rate and rate of rhodium loss. U.S. Pat. No. 8,404,903 is focused on the first two factors and mentions that the presence of CO in the recycle gas should be beneficial for stability of the phosphite ligand, but there is no mention of slowing or preventing rhodium loss. U.S. Pat. No. 8,404,903 is limited by the cooling temperature of the vaporizer condenser which may require substantial capital investment to achieve sufficiently low process fluid temperatures to achieve the desired CO partial pressure.

WO2016089602A1 expands on the process taught in U.S. Pat. No. 8,404,903 to add a CO-enriched gas to the recycle gas to increase the CO partial pressure above 16 psia while maintaining acceptable total pressures. The added CO primarily displaces inert gases in the recycle stream thus higher CO partial pressures are realized. This patent also teaches that high levels of H₂ are detrimental to catalyst life; Table 3 in particular shows that the effect of a low CO/H₂ ratio on catalyst stability.

WO2020240194 teaches to use a membrane unit to separate the syngas supply from the hydroformylation system to generate the CO-enriched stream to be fed to the strip gas vaporizer with the improvement that the H₂-enriched stream to be sent to the hydroformylation zone (combined with a CO purge from the vaporizer vent). This teaches a means to generate a “highly enriched CO stream” used in WO2016089602 for higher vaporizer CO partial pressures (at or above 15 psia (103 kPa)) and prefers the hydrogen partial pressure to be not more than 10 psi (69 kPa).

U.S. Pat. No. 8,404,903, WO2016089602A1, and WO2020240194 all involve using a recycle gas stream in the vaporizer and using a purge vent (e.g., stream 25 in WO2016089602A1) to control the composition of the stripping gas. In such systems, since the CO is largely insoluble in the liquid phase being removed from the vaporizer condenser, most of the CO is recycled thus it accumulates in the recycle gas (as taught in U.S. Pat. No. 8,404,903) and only small amounts of added CO are needed if WO2016089602A1 process is employed to further increase the CO partial pressure. All three are focused solely on controlling the CO partial pressure within the vaporizer and ignore the accumulation of H₂. In U.S. Pat. No. 8,404,903, at lower condenser temperatures, the CO fraction is going up but the CO/H₂ ratio is dropping (the relative amount of H₂ is increasing faster than the CO). WO2016089602A1 partially addresses this issue by directly adding a high CO-content stream to increase the CO content above a partial pressure of 16 psi but this necessitates a total pressure of well above 16 psia which is not optimal for higher molecular weight aldehydes.

As taught by van Leeuwen et al. (Organometallics, 14, 34 (1995)) and Rush et al. (Kinetics and Catalysis, 50, 557 (2009)), the reaction rate is positive order in H₂ partial pressure for phosphite-based catalysts thus the amount of H₂ present is generally kept high in the reaction zone up to the point where hydrogenation to alkanes become pronounced. There is no teaching on any detrimental effects on catalyst stability due to higher hydrogen content. Rush further teaches that there is no change in hydroformylation behavior beyond a monophosphite:rhodium ratio above 50:1, up to 250:1, but is silent on any benefit on catalyst life at higher ratios. Enhanced stability based on ligand:Rh ratio is taught in U.S. Pat. No. 4,277,627 for triphenylphosphine, for example, but only in relation to the hydroformylation reaction rather than in the vaporizer for phosphines.

Both U.S. Pat. No. 4,277,627 and WO2020112373 teach that olefin stabilizes rhodium-based hydroformylation catalysts; the latter focuses in particular on the vaporizer where the olefin concentration would be expected to be the lowest. However, WO2020112373 requires unreacted olefin to be present throughout the vaporizer which could represent olefin inefficiency and/or substantial olefin recycling processing. With highly reactive lower olefins such as ethylene, propylene, and 1-butene with highly active hydroformylation catalysts, the amount of unreacted olefin present in the vaporizer will be very low.

The ratio of CO and H₂ within the vaporizer stripping gas is a function of how much CO and H₂ comes in with the stream from the hydroformylation zone (which itself is a function of the CO and H₂ ratio in the syngas feed in the reaction zone), the purge vent, the vaporizer condenser temperature, the total pressure, and the amount of inert gases present in the incoming stream (e.g., methane, alkanes, etc.) as well as any added CO or H₂. For example, as the inerts content increase at constant total pressure, the CO content will likely become lower in the recycle gas stream. To compensate, the total pressure might be increased but this will negatively impact the aldehyde vaporization process, resulting in catalyst degradation. The alternative is to increase the recycle gas flow to purge more inerts but this increased flow risks catalyst entrainment losses and requires larger condensers and recycle blowers/compressors.

It has now been discovered that while higher CO partial pressures are desirable for catalyst stability, lower hydrogen partial pressures are also desirable for cases where the CO partial pressure is lower than 15 psia and where olefin concentrations are low. This scenario would be present in the vaporizer with lower molecular weight olefin processes, for example, where most if not all of the olefin has been converted (or vaporized away). Lower CO partial pressures may be present when the total pressure of the vaporizer is kept low to enhance the product vaporization at lower temperatures which is generally preferred for catalyst stability. Under these conditions, the CO partial pressure is sufficiently low that undesirable hydrogen-driven catalyst deactivation processes occur in the vaporizer.

In cases where it is not possible to have sufficiently high CO partial pressure to maintain stability due to equipment or other process limitations, we have found that higher ligand concentration and especially higher ligand-to-transition metal ratios can also be employed to mitigate catalyst stability when operating at less than 15 psi CO partial pressure.

In view of the deficiencies of the prior art, there remains a need for a means of separating aldehydes from a rhodium-organophosphite hydroformylation catalyst at lower total pressures while reducing the loss of rhodium and/or catalyst activity in situations where the catalyst stabilization by residual olefin and CO is substantially reduced.

### SUMMARY OF THE INVENTION

The process of the invention is such a continuous hydroformylation process comprising: (a) removing from a reactor a crude product; (b) sending the crude product to a vaporizer; (c) separating the crude product in the vaporizer to produce a catalyst-containing liquid stream and a gas phase stream; and (d) maintaining an average CO partial pressure in the vaporizer of less than 15 psia (103 kPa) with reduced H₂ content.

In one embodiment, the step of maintaining the average CO pressure in the vaporizer at less than 15 psia (103 kPa) comprises one or more of the following three processes:
1: (a) feeding a crude product stream comprising one or more products, one or more heavy by-products, a transition metal-organophosphite ligand complex catalyst, one or more unconverted reactants, and one or more inert lights into a vaporizer; and
   (b) removing from the vaporizer an overhead gas stream comprising one or more products, one or more unconverted reactants, one or more inert lights, hydrogen, carbon monoxide, and a portion of the heavy by-products, and feeding said overhead gas stream into a condenser; and
   (c) removing from the condenser a condenser overhead gas stream comprising one or more unconverted reactants and one or more inert lights; and
   (d) recycling at least a portion of said condenser overhead gas stream to the vaporizer and/or a reaction zone; and
   (e) introducing to the vaporizer, in addition to the condenser overhead gas stream, a gas stream comprising CO, such that the average CO partial pressure in the vaporizer is less than 15 psia (103 kPa);
      wherein the molar ratio of CO to H₂ in the gas stream being added is higher than the ratio present at step (b), and
      wherein the vaporizer total pressure, CO partial pressure, and H₂ partial pressure are controlled by the fraction of the condenser overhead gas stream recycled in step (d); and
   (f) removing as a tails stream from the vaporizer, a liquid recycle catalyst stream comprising the transition metal-organophosphite ligand complex catalyst and the balance of the heavy by-products.
2: (a) feeding a crude product stream comprising one or more products, one or more heavy by-products, a transition metal-organophosphite ligand complex catalyst, one or more unconverted reactants, and one or more inert lights into a depressurization zone wherein a gaseous purge is removed and the liquid phase is sent to a vaporizer;
   wherein the depressurization zone removes undissolved H₂ and optionally inert gases such that the amount of H₂ being introduced to the vaporizer is reduced to allow reduced total vaporizer pressure; and
   (b) removing from the vaporizer an overhead gas stream comprising one or more products, one or more unconverted reactants, one or more inert lights, hydrogen, carbon monoxide, and a portion of the heavy by-products, and feeding said overhead gas stream into a condenser; and
   (c) removing from the condenser a condenser overhead gas stream comprising one or more unconverted reactants and one or more inert lights; and
   (d) recycling at least a portion of said condenser overhead gas stream to the vaporizer and/or a reaction zone; and
   (e) introducing to the vaporizer, in addition to the condenser overhead gas stream, a gas stream comprising CO, such that the average CO partial pressure in the vaporizer is less than 15 psia (103 kPa);
   wherein the vaporizer total pressure, CO partial pressure, and H₂ partial pressure are controlled by the fraction of the condenser overhead gas stream recycled in step (d); and
   (f) removing as a tails stream from the vaporizer, a liquid recycle catalyst stream comprising the transition metal-organophosphite ligand complex catalyst and the balance of the heavy by-products.
3: (a) feeding a crude product stream comprising one or more products, one or more heavy by-products, a transition metal-organophosphite ligand complex catalyst, one or more unconverted reactants, and one or more inert lights into a vaporizer; and
   (b) removing from the vaporizer an overhead gas stream comprising one or more products, one or more unconverted reactants, one or more inert lights, hydrogen, carbon monoxide, and a portion of the heavy by-products, and feeding said overhead gas stream into a condenser; and
   (c) removing from the condenser a condenser overhead gas stream comprising one or more unconverted reactants and one or more inert lights; and
   (d) recycling at least a portion of said condenser overhead gas stream to the vaporizer and/or a reaction zone; and
   (e) introducing to the vaporizer, in addition to the condenser overhead gas stream, a gas stream comprising CO, such that the average CO partial pressure in the vaporizer is less than 15 psia (103 kPa);
      wherein the vaporizer total pressure, CO partial pressure, and H₂ partial pressure are controlled by the fraction of the condenser overhead gas stream recycled in step (d); and
   (f) removing as a tails stream from the vaporizer, a liquid recycle catalyst stream comprising the transition metal-organophosphite ligand complex catalyst and the balance of the heavy by-products.
      wherein the ligand: transition metal ratio of the crude product stream in step (a) is above 20:1, the transition metal concentration in the tails stream in step (f) is less than 100 ppm, or both.

For example, with scheme 1, an analyzer measures the CO content (or partial pressure) of the recycle gas (at step (c) or (d)) and if the CO is below a target value, a CO-enriched stream is added at step (e) (said stream having a CO:H₂ ratio higher than that of the recycle gas). This would cause the system pressure to increase so a pressure controller would increase the vaporizer vent to purge out some of the gas to maintain constant total pressure. All of this has the effect of increasing CO and reducing other components (namely H₂). The target CO partial pressure will be a function of equipment limitations such as total pressure, steam temperature (for the vaporizer), cooling water, and the pressure of the CO-enriched gas for step (e). The total pressure limit will be a function of the other gases such as inerts from feeds (e.g., N₂ from syngas or paraffins from olefin feed or inadvertent hydrogenation of olefins). The stripping gas flow rate is set such that the vaporizer temperature remains below a set limit (primarily driven by available steam and condenser cooling medium temperatures and avoidance of excessive heavies formation and catalyst degradation). The amount of heat input (and temperature) is set by the vaporizer feed-to-tails (“F/T”) ratio, again a function of the available steam and avoidance of undesired side reactions. The prior art teaches that higher CO partial pressure is preferred; it is not always economical to achieve those pressures but the current scheme which also reduces the H₂ content enables comparable catalyst stability. For example, higher condenser temperatures as taught in U.S. Pat. No. 8,404,903 may result in higher aldehyde losses via the purge thus represents a process efficiency loss and refrigeration units represent a high capital expense.

With scheme 2, the flash pot reduces the total amount of high pressure gases being introduced to the vaporizer as well as removing a substantial amount of H₂ which may be in excess of the amount of CO present. Hydrogen has a lower solubility than CO in the liquid matrix thus this enriches the CO relative to the H₂ in residual gases being introduced to the vaporization zone via the liquid phase. The flash pot is typically operated at a substantially elevated pressure compared to the vaporizer thus the condenser (not shown in FIG. 2) is better able to minimize aldehyde product losses. Removing a substantial portion of non-condensable gases enables easier operation of the vaporizer and reduces the amount of flow lost via the vaporizer condenser vent which operates at lower pressure than the flash pot. The reduced flow and lower total pressure facilitate a buildup of CO while keeping the H₂ concentration and vent losses low.

In Scheme 3, it has been surprisingly found that higher ligand:rhodium ratios not only give higher rates, but higher stability. Assuming the rate of rhodium loss is due to formation of catalytically inactive, multi-metal sites (thus likely positive order in rhodium concentration), a lower rhodium concentration should give slower inactivation at the cost of lower hydroformylation rates. However, combining the higher rate due to increased ligand concentration with lower starting rhodium concentration offsets the reduced rate due to lower rhodium concentration thus the two factors give the same rate but higher catalyst stability.

To implement scheme 3, higher ligand concentrations can be used up to the point of solubility issues (primarily found in the vaporizer bottoms recycle line such as stream 13 in FIG. 1). Changing the concentration of the phosphite ligand is generally simple to do either by adding additional ligand to increase the concentration or allowing the natural ligand decomposition and vaporization losses to continue without the conventional replenishments. Direct introduction of an oxidant (e.g., 02 or a peroxide) can be used as well but is generally not preferred. Likewise, reducing the rhodium concentration is generally not preferred since the costly rhodium would need to be recovered although it can be reduced slowly by the normal entrainment losses via the vaporizer and not performing the usual periodic rhodium replenishment charges. Thus, the preferred embodiment is to initiate the catalyst at high ligand:rhodium ratios such as above 20:1, preferably above 40:1 with rhodium concentrations below 100 ppm, and preferably below 60 ppm and most preferably below 40 ppm in the reaction zone.

Superatmospheric pressure in the vaporizer is normally avoided as a process condition for the vaporization of C₃ and higher aldehydes. Thus, it is surprising that even under the modest CO partial pressures in the superatmospheric pressure environment of the vaporizer, good transition metal-organophosphite catalyst stability is observed while simultaneously allowing removal of high boiling aldehydes and aldehyde condensation products at moderate temperatures. The lower H₂ concentration at these modest CO partial pressures is a key parameter to this discovery in addition to trading off higher rate with higher ligand: transition metal with lower transition metal levels to achieve comparable overall production rate with less transition metal loss.

### BRIEF DESCRIPTION OF THE DRAWINGS

FIG. 1 is a schematic flowsheet of one embodiment of the process of the present invention.

FIG. 2 is a schematic flowsheet of another embodiment of the process of the present invention.

### DETAILED DESCRIPTION OF THE INVENTION

A hydroformylation process comprises contacting CO, H₂, and at least one olefin under hydroformylation conditions sufficient to form at least one aldehyde product in the presence of a catalyst comprising, as components, a transition metal and a hydrolyzable ligand. Optional process components include an amine and/or water.

All references to the Periodic Table of the Elements and the various groups therein are to the version published in the CRC Handbook of Chemistry and Physics, 72nd Ed. (1991-1992) CRC Press, at page I-11.

Unless stated to the contrary, or implicit from the context, all parts and percentages are based on weight and all test methods are current as of the filing date of this application. For purposes of United States patent practice, the contents of any referenced patent, patent application or publication are incorporated by reference in their entirety (or its equivalent US version is so incorporated by reference) especially with respect to the disclosure of definitions (to the extent not inconsistent with any definitions specifically provided in this disclosure) and general knowledge in the art.

As used herein, “a,” “an,” “the,” “at least one,” and “one or more” are used interchangeably. The terms “comprises,” “includes,” and variations thereof do not have a limiting meaning where these terms appear in the description and claims. Thus, for example, an aqueous composition that includes particles of “a” hydrophobic polymer can be interpreted to mean that the composition includes particles of “one or more” hydrophobic polymers.

Also herein, the recitations of numerical ranges by endpoints include all numbers subsumed in that range (e.g., 1 to 5 includes 1, 1.5, 2, 2.75, 3, 3.80, 4, 5, etc.). For the purposes of the invention, it is to be understood, consistent with what one of ordinary skill in the art would understand, that a numerical range is intended to include and support all possible sub ranges that are included in that range. For example, the range from 1 to 100 is intended to convey from 1.01 to 100, from 1 to 99.99, from 1.01 to 99.99, from 40 to 60, from 1 to 55, etc. Also herein, the recitations of numerical ranges and/or numerical values, including such recitations in the claims, can be read to include the term “about.” In such instances the term “about” refers to numerical ranges and/or numerical values that are substantially the same as those recited herein.

As used herein, the terms “ppm” and “ppmw” mean parts per million by weight. When referring to concentration of transition metals in catalyst solution, it refers to the weight of the metal (not including ligands if any) divided by the mass of rest of the solution including solvents, free ligand, reactants, and impurities.

For purposes of this invention, the term “hydrocarbon” is contemplated to include all permissible compounds having at least one hydrogen and one carbon atom. Such permissible compounds may also have one or more heteroatoms. In a broad aspect, the permissible hydrocarbons include acyclic (with or without heteroatoms) and cyclic, branched and unbranched, carbocyclic and heterocyclic, aromatic and nonaromatic organic compounds that can be substituted or unsubstituted.

As used herein, the term “substituted” is contemplated to include all permissible substituents of organic compounds unless otherwise indicated. In a broad aspect, the permissible substituents include acyclic and cyclic, branched and unbranched, carbocyclic and heterocyclic, aromatic and nonaromatic substituents of organic compounds. Illustrative substituents include, for example, alkyl, alkyloxy, aryl, aryloxy, hydroxyalkyl, aminoalkyl, in which the number of carbons can range from 1 to 20 or more, preferably from 1 to 12, as well as hydroxy, halo, and amino. The permissible substituents can be one or more and the same or different for appropriate organic compounds. This invention is not intended to be limited in any manner by the permissible substituents of organic compounds.

The terms “reaction fluid,” “reaction medium” and “catalyst solution” are used interchangeably herein, and may include, but are not limited to, a mixture comprising: (a) a metal-organophosphorous ligand complex catalyst, (b) free organophosphorous ligand, (c) aldehyde product formed in the reaction, (d) unreacted reactants, (e) a solvent for said metal-organophosphorous ligand complex catalyst and said free organophosphorous ligand, and, optionally, (f) one or more phosphorus acidic compounds formed in the reaction (which may be homogeneous or heterogeneous, and these compounds include those adhered to process equipment surfaces). The reaction fluid can encompass, but is not limited to, (a) a fluid in a reaction zone, (b) a fluid stream on its way to a separation zone, (c) a fluid in a separation zone, (d) a recycle stream, (e) a fluid withdrawn from a reaction zone or separation zone, (f) a withdrawn fluid being treated with an aqueous buffer solution, (g) a treated fluid returned to a reaction zone or separation zone, (h) a fluid in an external cooler, and (i) ligand decomposition products and their salts.

“Hydrolyzable phosphorous ligands” are trivalent phosphorous ligands that contain at least one P—Z bond wherein Z is oxygen, nitrogen, chlorine, fluorine or bromine. Examples include, but are not limited to, phosphites, phosphino-phosphites, bisphosphites, phosphonites, bisphosphonites, phosphinites, phosphoramidites, phosphino-phosphoramidites, bisphosphoramidites, fluorophosphites, and the like. The ligands may include chelate structures and/or may contain multiple P-Z moieties such as polyphosphites, polyphosphoramidites, etc. and mixed P-Z moieties such as phosphite-phosphoramidites, flurophosphite-phosphites, and the like.

The term “complex” as used herein means a coordination compound formed by the union of one or more electronically rich molecules or atoms (i.e., ligand) with one or more electronically poor molecules or atoms (i.e., transition metal). For example, the organophosphorous ligand employable herein possesses one phosphorus (III) donor atom having one unshared pair of electrons, which is capable of forming a coordinate covalent bond with the metal. A polyorganophosphorous ligand employable herein possesses two or more phosphorus (III) donor atoms, each having one unshared pair of electrons, each of which is capable of forming a coordinate covalent bond independently or possibly in concert (for example, via chelation) with the transition metal. Carbon monoxide can also be present and complexed with the transition metal. The ultimate composition of the complex catalyst may also contain an additional ligand(s) such as described above, for example, hydrogen, mono-olefin, or an anion satisfying the coordination sites or nuclear charge of the metal.

For the purposes of this invention, the terms “heavy by-products” and “heavies” are used interchangeably and refer to hydroformylation process liquid by-products that have a normal boiling point that is at least 25° C. above the normal boiling point of the desired product of the process. In a hydroformylation reaction, for example, where the reactant comprises one or more olefins, the desired product frequently comprises one or more isomeric aldehydes, as well as heavies.

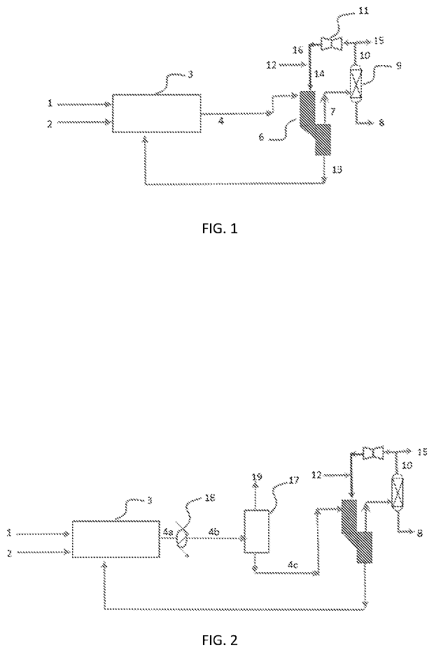

For the purposes of this invention, the terms “feed to tails” and “feed to tails ratio” are used interchangeably and refer to the mass of reaction fluid entering the separation zone relative to the mass of concentrated effluent (vaporizer tails) leaving the bottom of the separation zone and returning to the first hydroformylation reactor. Referring to FIG. 1, this is the mass ratio of stream 4 to stream 13. “Feed to tails” is an indicator of the rate at which volatiles, such as aldehyde product, are removed from the reaction fluid. For example, a “feed to tails ratio” of 2, means that the weight of reaction fluid entering the separation zone is two times greater than the weight of the concentrated effluent returned to the first reactor.

For purposes of this invention, the terms decompression zone, “knock-out pot”, “knock-out vessel” and “flash vessel” are used interchangeably and refer to low pressure sections between the reaction zone and the vaporizer. The flash vessel allows the reaction fluid to rapidly degas and facilitates control of the vaporizer partial pressures. The flash vessel also removes any entrained gas bubbles from the liquid phase that may be transporting more H₂ than what would simply be dissolved in the liquid phase from the hydroformylation reaction zone. Such vessels are typically maintained at pressures and temperatures well below those established in the hydroformylation reactors.

For the purposes of this invention, the term “lights” refers to materials that have a normal boiling point of 25° C. or less at atmospheric pressure. As used herein, the term “inert lights” or “light inerts” refers to lights that are essentially unreactive in the process. “Reactive lights” shall refer to lights that are reactive to a significant degree in the process. As an example, in a hydroformylation process, reactive lights include carbon monoxide and hydrogen; while inert lights include alkanes, such as alkanes that are present in the olefinic feed to the reaction, and other inert gases such as nitrogen.

“Essentially isobarically” and like terms mean at essentially constant pressure or within a pressure difference of 1 bar (100 kPa) or less, preferably 0.5 bar (50 kPa) or less. In other words, in one embodiment of the invention the maximum pressure difference across the product phase stripper and the product condenser is 1 bar (100 kPa) or less, preferably 0.5 bar (50 kPa) or less.

The terms “vaporizer,” “stripping gas vaporizer,” “stripper” and “product phase stripper” are used herein interchangeably, and refer to a separation device that employs stripping gas to aid in the separation of the components of the product-containing stream from the product.

As used herein, the term “average CO partial pressure” means the average carbon monoxide partial pressure determined at the vapor outlet of the vaporizer over at least a 10 minute period at steady state operation. Determining mole % of CO in a gas composition using gas chromatography (GC) is well known; CO partial pressure is then calculated by measuring total pressure and using Raoult's Law.

As used herein, the term “average H₂ partial pressure” means the average hydrogen partial pressure determined at the vapor outlet of the vaporizer over at least a 10 minute period at steady state operation. Determining mole % of H₂ in a gas composition using gas chromatography (GC) is well known; hydrogen partial pressure is then calculated by measuring total pressure and using Raoult's Law.

Hydrogen and carbon monoxide may be obtained from any suitable source, including petroleum cracking and refinery operations. Syngas mixtures are a preferred source of hydrogen and CO for the hydroformylation reaction zone.

Syngas (from synthesis gas) is the name given to a gas mixture that contains varying amounts of CO and H₂. Production methods are well known. Hydrogen and CO typically are the main components of syngas, but syngas may contain CO₂ and inert gases such as qN₂ and Ar. The molar ratio of H₂ to CO varies greatly but generally ranges from 1:100 to 100:1 and preferably between 1:10 and 10:1. Syngas is commercially available and is often used as a fuel source or as an intermediate for the production of other chemicals. The most preferred H₂:CO molar ratio for chemical production is between 3:1 and 1:3 and usually is targeted to be between about 1:2 and 2:1 for most hydroformylation applications.

The substituted or unsubstituted olefinic reactants that may be employed in the hydroformylation process include both optically active (prochiral and chiral) and non-optically active (achiral) olefinic unsaturated compounds containing from 2 to 40, preferably 3 to 30, carbon atoms, more preferably from 4 to 20 carbon atoms. These compounds are described in detail in U.S. Pat. No. 7,863,487. Such olefinic unsaturated compounds can be terminally or internally unsaturated and be of straight-chain, branched chain or cyclic structures, as well as olefin mixtures, such as obtained from the dimerization of mixed butenes, the oligomerization of propene, butene, isobutene, etc. (such as so called dimeric, trimeric or tetrameric propylene and the like, as disclosed, for example, in U.S. Pat. Nos. 4,518,809 and 4,528,403).

Prochiral and chiral olefins useful in the asymmetric hydroformylation can be employed to produce enantiomeric aldehyde mixtures. Illustrative optically active or prochiral olefinic compounds useful in asymmetric hydroformylation are described, for example, in U.S. Pat. Nos. 4,329,507, 5,360,938 and 5,491,266.

A solvent advantageously is employed in the hydroformylation process. Any suitable solvent that does not unduly interfere with the hydroformylation process can be used. By way of illustration, suitable solvents for transition metal catalyzed hydroformylation processes include those disclosed, for example, in U.S. Pat. Nos. 3,527,809; 4,148,830; 5,312,996; and 5,929,289. In rhodium catalyzed hydroformylation processes, it may be preferred to employ, as a primary solvent, aldehyde compounds corresponding to the aldehyde products desired to be produced and/or higher boiling aldehyde liquid condensation by-products, for example, as might be produced in situ during the hydroformylation process, as described for example in U.S. Pat. Nos. 4,148,380 and 4,247,486. The primary solvent will normally eventually comprise both aldehyde products and higher boiling aldehyde liquid condensation by-products (“heavies”), due to the nature of the continuous process. The amount of solvent is not especially critical and need only be sufficient to provide the reaction medium with the desired amount of transition metal concentration. Typically, the amount of solvent ranges from about 5 percent to about 95 percent by weight, based on the total weight of the reaction fluid. Mixtures of solvents may be employed.

Illustrative metal-organophosphorous ligand complexes employable in such hydroformylation reactions include metal-organophosphorous ligand complex catalysts. These catalysts, as well as methods for their preparation, are well known in the art and include those disclosed in the patents mentioned herein. In general, such catalysts may be preformed or formed in situ and comprise metal in complex combination with an organophosphorous ligand, carbon monoxide and optionally hydrogen. The exact structure of the catalyst is not known.

The metal-organophosphorous ligand complex catalyst can be optically active or non-optically active. The metals can include Group 8, 9 and 10 metals selected from rhodium (Rh), cobalt (Co), iridium (Ir), ruthenium (Ru), iron (Fe), nickel (Ni), palladium (Pd), platinum (Pt), osmium (Os) and mixtures thereof, with the preferred metals being rhodium, cobalt, iridium and ruthenium, more preferably rhodium, cobalt and ruthenium, especially rhodium. Mixtures of these metals may be used. The permissible organophosphorous ligands that make up the metal-organophosphorous ligand complexes and free organophosphorous ligand include mono-, di-, tri- and higher polyorganophosphorus ligands. Mixtures of ligands may be employed in the metal-organophosphorous ligand complex catalyst and/or free ligand, and such mixtures may be the same or different. In one embodiment of the invention, a mixture of monoorganophosphite and organopolyphosphite, e.g., bisphosphite, ligands can be employed.

The organophosphorous compounds that may serve as the ligand of the metal-organophosphorous ligand complex catalyst and/or free ligand may be of the achiral (optically inactive) or chiral (optically active) type and are well known in the art. Achiral organophosphorous ligands are preferred.

Among the organophosphorous ligands that may serve as the ligand of the metal-organophosphorous ligand complex catalyst are monoorganophosphite, diorganophosphite, triorganophosphite and organopolyphosphite compounds. Such organophosphorous ligands and methods for their preparation are well known in the art.

Representative monoorganophosphites may include those having the formula:

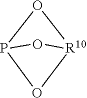

wherein R¹⁰ represents a substituted or unsubstituted trivalent hydrocarbon radical containing from 4 to 40 carbon atoms or greater, such as trivalent acyclic and trivalent cyclic radicals, e.g., trivalent alkylene radicals such as those derived from 1,2,2-trimethylolpropane and the like, or trivalent cycloalkylene radicals such as those derived from 1,3,5-trihydroxycyclohexane and the like. Such monoorganophosphites may be found described in greater detail, for example, in U.S. Pat. No. 4,567,306.

Representative diorganophosphites may include those having the formula:

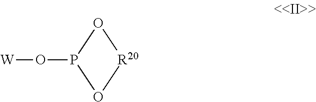

wherein R²⁰ represents a substituted or unsubstituted divalent hydrocarbon radical containing from 4 to 40 carbon atoms or greater and W represents a substituted or unsubstituted monovalent hydrocarbon radical containing from 1 to 18 carbon atoms or greater.

Representative substituted and unsubstituted monovalent hydrocarbon radicals represented by W in the above Formula (II) include alkyl and aryl radicals, while representative substituted and unsubstituted divalent hydrocarbon radicals represented by R²⁰ include divalent acyclic radicals and divalent aromatic radicals. Illustrative divalent acyclic radicals include, for example, alkylene, alkylene-oxy-alkylene, alkylene-S-alkylene, cycloalkylene radicals, and, alkylene-NR²⁴-alkylene wherein R²⁴ is hydrogen or a substituted or unsubstituted monovalent hydrocarbon radical, e.g., an alkyl radical having 1 to 4 carbon atoms. The more preferred divalent acyclic radicals are the divalent alkylene radicals such as disclosed more fully, for example, in U.S. Pat. Nos. 3,415,906 and 4,567,302 and the like. Illustrative divalent aromatic radicals include, for example, arylene, bisarylene, arylene-alkylene, arylene-alkylene-arylene, arylene-oxy-arylene, arylene-NR²⁴-arylene wherein R²⁴ is as defined above, arylene-S-arylene, arylene-S-alkylene and the like. More preferably R²⁰ is a divalent aromatic radical such as disclosed more fully, for example, in U.S. Pat. Nos. 4,599,206, 4,717,775, 4,835,299, and the like.

Representative of a more preferred class of diorganophosphites are those of the formula:

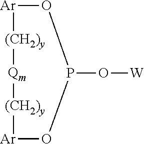

wherein W is as defined above, each Ar is the same or different and represents a substituted or unsubstituted aryl radical, each y is the same or different and is a value of 0 or 1, Q represents a divalent bridging group selected from —C(R³³)₂—, —O—, —S—, —NR²⁴—, Si(R³⁵)₂ and —CO—, wherein each R³³ is the same or different and represents hydrogen, an alkyl radical having from 1 to 12 carbon atoms, phenyl, tolyl, and anisyl, R²⁴ is as defined above, each R³⁵ is the same or different and represents hydrogen or a methyl radical, and m has a value of 0 or 1. Such diorganophosphites are described in greater detail, for example, in U.S. Pat. Nos. 4,599,206, 4,717,775, and 4,835,299.

Representative triorganophosphites may include those having the formula:

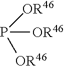

wherein each R⁴⁶ is the same or different and is a substituted or unsubstituted monovalent hydrocarbon radical e.g., an alkyl, cycloalkyl, aryl, alkaryl and aralkyl radicals that may contain from 1 to 24 carbon atoms. Illustrative triorganophosphites include, for example, trialkyl phosphites, dialkylaryl phosphites, alkyldiaryl phosphites, triaryl phosphites, and the like, such as, for example, trimethyl phosphite, triethyl phosphite, butyldiethyl phosphite, dimethylphenyl phosphite, triphenyl phosphite, trinaphthyl phosphite, bis(3,6,8-tri-t-butyl-2-naphthyl)methylphosphite, bis(3,6,8-tri-t-butyl-2-naphthyl)cyclohexylphosphite, tris(3,6-di-t-butyl-2-naphthyl)phosphite, bis(3,6,8-tri-t-butyl-2-naphthyl)phenylphosphite, and bis(3,6,8-tri-t-butyl-2-naphthyl)(4-sulfonylphenyl)phosphite, and the like. The most preferred triorganophosphite is triphenylphosphite. Such triorganophosphites are described in greater detail, for example, in U.S. Pat. Nos. 3,527,809 and 5,277,532.

Representative organopolyphosphites contain two or more tertiary (trivalent) phosphorus atoms and may include those having the formula:

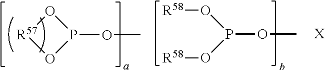

wherein X represents a substituted or unsubstituted n-valent organic bridging radical containing from 2 to 40 carbon atoms, each R⁵⁷ is the same or different and represents a divalent organic radical containing from 4 to 40 carbon atoms, each R⁵⁸ is the same or different and represents a substituted or unsubstituted monovalent hydrocarbon radical containing from 1 to 24 carbon atoms, a and b can be the same or different and each have a value of 0 to 6, with the proviso that the sum of a+b is 2 to 6 and n equals a+b. It is to be understood that when a has a value of 2 or more, each R⁵⁷ radical may be the same or different. Each R⁵⁸ radical may also be the same or different in any given compound.

Representative n-valent (preferably divalent) organic bridging radicals represented by X and representative divalent organic radicals represented by R⁵⁷ above, include both acyclic radicals and aromatic radicals, such as alkylene, alkylene-Qₘ-alkylene, cycloalkylene, arylene, bisarylene, arylene-alkylene, and arylene-(CH₂)_{y}-Qₘ-(CH₂)_{y}-arylene radicals, and the like, wherein each Q, y and m are as defined above in Formula (III). The more preferred acyclic radicals represented by X and R⁵⁷ above are divalent alkylene radicals, while the more preferred aromatic radicals represented by X and R⁵⁷ above are divalent arylene and bisarylene radicals, such as disclosed more fully, for example, in U.S. Pat. Nos. 4,769,498; 4,774,361; 4,885,401; 5,179,055; 5,113,022; 5,202,297; 5,235,113; 5,264,616; 5,364,950 and 5,527,950. Representative preferred monovalent hydrocarbon radicals represented by each R⁵⁸ radical above include alkyl and aromatic radicals.

Illustrative preferred organopolyphosphites may include bisphosphites such as those of Formulas (VI) to (VIII) below:

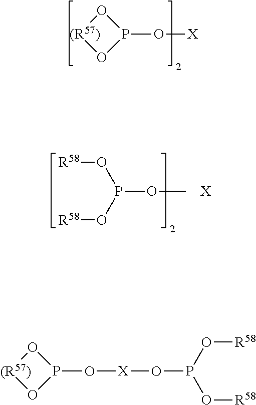

wherein each R⁵⁷, R⁵⁸ and X of Formulas (VI) to (VIII) are the same as defined above for Formula (V). Preferably each R⁵⁷ and X represents a divalent hydrocarbon radical selected from alkylene, arylene, arylene-alkylene-arylene, and bisarylene, while each R⁵⁸ radical represents a monovalent hydrocarbon radical selected from alkyl and aryl radicals. Organophosphite ligands of such Formulas (V) to (VIII) may be found disclosed, for example, in U.S. Pat. Nos. 4,668,651; 4,748,261; 4,769,498; 4,774,361; 4,885,401; 5,113,022; 5,179,055; 5,202,297; 5,235,113; 5,254,741; 5,264,616; 5,312,996; 5,364,950; and 5,391,801.

R¹⁰, R²⁰, R⁴⁶, R⁵⁷, R⁵⁸, Ar, Q, X, m, and y in Formulas (VI) to (VIII) are as defined above. Most preferably X represents a divalent aryl-(CH₂)_{y}-(Q)ₘ-(CH₂)_{y}-aryl radical wherein each y individually has a value of 0 or 1; m has a value of 0 or 1 and Q is —O—, —S— or —C(R³⁵)₂— where each R³⁵ is the same or different and represents hydrogen or a methyl radical. More preferably each alkyl radical of the above defined R⁵⁸ groups may contain from 1 to 24 carbon atoms and each aryl radical of the above-defined Ar, X, R⁵⁷ and R⁵⁸ groups of the above Formulas (VI) to (VIII) may contain from 6 to 18 carbon atoms and said radicals may be the same or different, while the preferred alkylene radicals of X may contain from 2 to 18 carbon atoms and the preferred alkylene radicals of R⁵⁷ may contain from 5 to 18 carbon atoms. In addition, preferably the divalent Ar radicals and divalent aryl radicals of X of the above formulas are phenylene radicals in which the bridging group represented by —(CH₂)_{y}-(Q)ₘ-(CH₂)_{y}— is bonded to said phenylene radicals in positions that are ortho to the oxygen atoms of the formulas that connect the phenylene radicals to their phosphorus atom of the formulae. It is also preferred that any substituent radical when present on such phenylene radicals be bonded in the para and/or ortho position of the phenylene radicals in relation to the oxygen atom that bonds the given substituted phenylene radical to its phosphorus atom.

Any of the R¹⁰, R²⁰, R⁵⁷, R⁵⁸, W, X, Q and Ar radicals of such organophosphites of Formulas (I) to (VIII) above may be substituted if desired, with any suitable substituent containing from 1 to 30 carbon atoms that does not unduly adversely affect the desired result of the process of this invention. Substituents that may be on said radicals in addition to corresponding hydrocarbon radicals such as alkyl, aryl, aralkyl, alkaryl and cyclohexyl substituents, may include for example silyl radicals such as —Si(R³⁵)₃; amino radicals such as —N(R¹⁵)₂; phosphine radicals such as -aryl-P(R¹⁵)₂; acyl radicals such as —C(O)R¹⁵ acyloxy radicals such as —OC(O)R¹⁵; amido radicals such as —CON(R¹⁵)₂ and —N—(R¹⁵)COR¹⁵; sulfonyl radicals such as —SO₂R¹⁵, alkoxy radicals such as —OR¹⁵; sulfinyl radicals such as —SOR¹⁵, phosphonyl radicals such as —P(O)(R¹⁵)₂, as well as halo, nitro, cyano, trifluoromethyl, hydroxy radicals and the like, wherein each R¹⁵ radical individually represents the same or different monovalent hydrocarbon radical having from 1 to 18 carbon atoms (e.g., alkyl, aryl, aralkyl, alkaryl and cyclohexyl radicals), with the proviso that in amino substituents such as —N(R¹⁵)₂ each R¹⁵ taken together can also represent a divalent bridging group that forms a heterocyclic radical with the nitrogen atom, and in amido substituents such as —C(O)N(R¹⁵)₂ and —N(R¹⁵)COR¹⁵ each R¹⁵ bonded to N can also be hydrogen. It is to be understood that any of the substituted or unsubstituted hydrocarbon radicals groups that make up a particular given organophosphite may be the same or different.

More specifically illustrative substituents include primary, secondary and tertiary alkyl radicals such as methyl, ethyl, n-propyl, isopropyl, butyl, sec-butyl, t-butyl, neo-pentyl, n-hexyl, amyl, sec-amyl, t-amyl, iso-octyl, decyl, octadecyl, and the like; aryl radicals such as phenyl, naphthyl, and the like; aralkyl radicals such as benzyl, phenylethyl, triphenylmethyl, and the like; alkaryl radicals such as tolyl, xylyl, and the like; alicyclic radicals such as cyclopentyl, cyclohexyl, 1-methylcyclohexyl, cyclooctyl, cyclohexylethyl, and the like; alkoxy radicals such as methoxy, ethoxy, propoxy, t-butoxy, —OCH₂CH₂OCH₃, —O(CH₂ CH₂)₂OCH₃, —O(CH₂CH₂)₃OCH₃, and the like; aryloxy radicals such as phenoxy and the like; as well as silyl radicals such as —Si(CH₃)₃, —Si(OCH₃)₃, —Si(C₃H₇)₃, and the like; amino radicals such as —NH₂, —N(CH₃)₂, —NHCH₃, —NH(C₂H₅), and the like; arylphosphine radicals such as —P(C₆H₅)₂, and the like; acyl radicals such as —C(O)CH₃, —C(O)C₂H₅, —C(O)C₆H₅, and the like; carbonyloxy radicals such as —C(O)OCH₃, and the like; oxycarbonyl radicals such as —O(CO)C₆H₅ and the like; amido radicals such as —CONH₂, —CON(CH₃)₂, —NHC(O)CH₃, and the like; sulfonyl radicals such as —S(O)₂C₂H₅ and the like; sulfinyl radicals such as —S(O)CH₃ and the like; sulfidyl radicals such as —SCH₃, —SC₂H₅, —SC₆H₅, and the like; phosphonyl radicals such as —P(O)(C₆H₅)₂, —P(O)(CH₃)₂, —P(O)(C₂H₅)₂, —P(O)(C₃H₇)₂, —P(O)(C₄H₉)₂, —P(O)(C₆H₁₃)₂, —P(O)CH₃(C₆H₅), —P(O)(H)(C₆H₅), and the like.

Specific illustrative examples of such organophosphite ligands include the following: tris(2,4-di-t-butylphenyl)phosphite, 2-t-butyl-4-methoxyphenyl(3,3′-di-t-butyl-5,5′-dimethoxy-1,1′-biphenyl-2,2′-diyl)phosphite, methyl(3,3′-di-t-butyl-5,5′-dimethoxy-1,1′-biphenyl-2,2′-diyl)phosphite, 6,6′-[[3,3′-bis(1,1-dimethylethyl)-5,5′-dimethoxy-[1,1′-biphenyl]-2,2′-diy 1]bis(oxy)]bis-dibenzo[d,f][1,3,2]dioxaphosphepin, 6,6′-[[3,3′,5,5′-tetrakis(1,1-dimethylethyl)-1,1′-biphenyl]-2,2′-diyl]bis(oxy)]bis-dibenzo[d,f][1,3,2]-dioxaphosphepin, (2R,4R)-di[2,2′-(3,3′, 5,5′-tetrakis-tert-butyl-1,1-biphenyl)]-2,4-pentyldiphosphite, (2R, 4R)di[2,2′-(3,3′-di-tert-butyl-5,5′-dimethoxy-1,1′-biphenyl)]-2,4-pentyldiphosphite, 2-[[2-[[4,8-bis(1,1-dimethylethyl), 2,10-dimethoxydibenzo-[d,f][1,3,2]dioxophosphepin-6-yl]oxy]-3-(1,1-dimethylethyl)-5-methoxyphenyl]methyl]-4-methoxy, methylenedi-2,1-phenylene tetrakis[2,4-bis(1,1-dimethylethyl)phenyl]ester of phosphorous acid, and [1,1′-biphenyl]-2,2′-diyl tetrakis[2-(1,1-dimethylethyl)-4-methoxyphenyl]ester of phosphorous acid.

In one embodiment, the organophosphite ligand comprises an organobisphosphite ligand. In one embodiment, the ligand is a bidentate phosphoramidite ligand, such as a bidentate phosphoramidite ligand of the class disclosed in, e.g., WO 00/56451 A1.

The metal-organophosphorous ligand complex catalysts may be in homogeneous or heterogeneous form. For instance, preformed rhodium hydrido-carbonyl-organophosphorous ligand catalysts may be prepared and introduced into a hydroformylation reaction mixture. More preferably, the rhodium-organophosphorous ligand complex catalysts can be derived from a rhodium catalyst precursor that may be introduced into the reaction medium for in situ formation of the active catalyst. For example, rhodium catalyst precursors such as rhodium dicarbonyl acetylacetonate, Rh₂O₃, Rh₄(CO)₁₂, Rh₆(CO)₁₆, Rh(NO₃)₃, and the like may be introduced into the reaction mixture along with the organophosphorous ligand for the in situ formation of the active catalyst. In a preferred embodiment, rhodium dicarbonyl acetylacetonate is employed as a rhodium precursor and reacted in the presence of a solvent with the organophosphorous ligand to form a catalytic rhodium-organophosphorous ligand complex precursor that is introduced into the reactor along with excess (free) organophosphorous ligand for the in situ formation of the active catalyst. In any event, it is sufficient that carbon monoxide, hydrogen and the organophosphorous ligand are all ligands that are capable of being complexed with the metal and that an active metal-organophosphorous ligand catalyst is present in the reaction mixture under the conditions used in the hydroformylation reaction. Carbonyl and organophosphorous ligands may be complexed to the rhodium either prior to or in situ during the hydroformylation process.

By way of illustration, a preferred catalyst precursor composition consists essentially of a solubilized rhodium carbonyl organophosphite ligand complex precursor, a solvent and, optionally, free organophosphite ligand. The preferred catalyst precursor composition can be prepared by forming a solution of rhodium dicarbonyl acetylacetonate, an organic solvent and a organophosphite ligand. The organophosphorous ligand readily replaces one of the carbonyl ligands of the rhodium acetylacetonate complex precursor as witnessed by the evolution of carbon monoxide gas.

Accordingly, the metal-organophosphorus ligand complex catalyst advantageously comprises the metal complexed with carbon monoxide and an organophosphorous ligand, said ligand being bonded (complexed) to the metal in a chelated and/or non-chelated fashion.

Mixtures of catalysts can be employed. The amount of metal-organophosphorous ligand complex catalyst present in the reaction fluid need only be that minimum amount necessary to provide the given metal concentration desired to be employed and that will furnish the basis for at least the catalytic amount of metal necessary to catalyze the particular hydroformylation process involved such as disclosed, for example, in the above-mentioned patents. In general, catalytic metal, e.g., rhodium, concentrations in the range of from 5 ppmw to 100 ppmw, calculated as free metal in the reaction medium, should be sufficient for most processes, while it is generally preferred to employ from 10 to 80 ppmw of metal, and more preferably from 20 to 60 ppmw of metal.

In addition to the metal-organophosphorous ligand complex catalyst, free organophosphorous ligand (i.e., ligand that is not complexed with the metal) may also be present in the reaction medium. The free organophosphorous ligand may correspond to any of the above-defined organophosphorous ligands discussed above. It is preferred that the free organophosphorous ligand be the same as the organophosphorous ligand of the metal-organophosphorous ligand complex catalyst employed. However, such ligands need not be the same in any given process. The hydroformylation process of this invention may involve from 0.1 moles or less to 100 moles or higher of free organophosphorous ligand per mole of metal in the reaction medium. Preferably, the hydroformylation process is carried out in the presence of from 1 to 50 moles of organophosphorous ligand per mole of metal present in the reaction medium. More preferably, for organopolyphosphites, from 1.1 to 4 moles of organopolyphosphite ligand are employed per mole of metal. Said amounts of organophosphorous ligand are the sum of both the amount of organophosphorous ligand that is bound (complexed) to the metal present and the amount of free organophosphorous ligand present. If desired, additional organophosphorous ligand can be supplied to the reaction medium of the hydroformylation process at any time and in any suitable manner, e.g., to maintain a predetermined level of free ligand in the reaction medium.

The use of an aqueous buffer solution, such as in an extraction system, to prevent and/or lessen hydrolytic degradation of an organophosphite ligand and deactivation of a metal-organophosphite ligand complex is well-known and is disclosed, e.g., in U.S. Pat. Nos. 5,741,942 and 5,741,944. Mixtures of buffers may be employed.

Optionally, an organic nitrogen compound may be added to the hydroformylation reaction fluid to scavenge the acidic hydrolysis by-products formed upon hydrolysis of the organophosphorous ligand, as taught, for example, in U.S. Pat. Nos. 4,567,306 and 5,731,472. Such organic nitrogen compounds may be used to react with and to neutralize the acidic compounds by forming conversion product salts therewith, thereby preventing the catalytic metal from complexing with the acidic hydrolysis by-products and thus helping to protect the activity of the catalyst while it is present in the reaction zone under reaction conditions.

Optionally, polymer additives can be used to help mitigate catalyst instability particularly in vaporization zones. Examples of these polymers are given in U.S. Pat. Nos. 4,774,361 and 11,111,198.

The hydroformylation process, and conditions for its operation, are well known. The hydroformylation process may be asymmetric or non-asymmetric, the preferred process being non-asymmetric, and may be conducted in any batch, continuous or semi-continuous fashion and may involve any catalyst liquid and/or gas recycle operation desired.

The hydroformylation reaction conditions employed will be governed by the type of aldehyde product desired. For instance, the total gas pressure of hydrogen, carbon monoxide and olefin starting compound of the hydroformylation process may range from 1 to 69,000 kPa. In general, however, it is preferred that the process be operated at a total gas pressure of hydrogen, carbon monoxide and olefin starting compound of less than 14,000 kPa and more preferably less than 3,400 kPa. The minimum total pressure is limited predominantly by the amount of reactants necessary to obtain a desired rate of reaction. More specifically, the carbon monoxide partial pressure of the hydroformylation process is preferably from 1 to 6,900 kPa, and more preferably from 21 to 5,500 kPa, while the hydrogen partial pressure is preferably from 34 to 3,400 kPa and more preferably from 69 to 2,100 kPa. In general, the molar ratio of gaseous H₂:CO may range from 1:10 to 100:1 or higher, the more preferred molar ratio being from 1:10 to 10:1.

In general, the hydroformylation process may be conducted at any operable reaction temperature. Advantageously, the hydroformylation process is conducted at a reaction temperature from −25° C. to 200° C., preferably from 50° C. to 120° C.

The hydroformylation process may be carried out using one or more suitable reactors such as, for example, a fixed bed reactor, a fluid bed reactor, a continuous stirred tank reactor (CSTR) or a slurry reactor. The optimum size and shape of the catalysts will depend on the type of reactor used. The reaction zone employed may be a single vessel or may comprise two or more discrete vessels.

The hydroformylation process of this invention may be conducted in one or more steps or stages. The exact number of reaction steps or stages will be governed by the best compromise between capital costs and achieving high catalyst selectivity, activity, lifetime and ease of operability, as well as the intrinsic reactivity of the starting materials in question and the stability of the starting materials and the desired reaction product to the reaction conditions.

In one embodiment, the hydroformylation process useful in this invention may be carried out in a multistaged reactor such as described, for example, in U.S. Pat. No. 5,728,893. Such multistaged reactors can be designed with internal, physical barriers that create more than one theoretical reactive stage per vessel.

It is generally preferred to carry out the hydroformylation process in a continuous manner. Continuous hydroformylation processes are well known in the art; the most preferred hydroformylation process comprises a continuous liquid catalyst recycle process. Suitable liquid catalyst recycle procedures are disclosed, for example, in U.S. Pat. Nos. 4,668,651; 4,774,361; 5,102,505 and 5,110,990.

FIG. 1 illustrates an integrated hydroformylation process of the invention. With reference to FIG. 1, an olefin feed stream 1 comprising one or more olefinic compounds and optionally one or more inert lights is fed into a hydroformylation reactor system 3 comprising one or more hydroformylation reactors (Oxo reactors). Concurrently, a gaseous feed stream 2 comprising carbon monoxide, hydrogen and optionally one or more gaseous inerts is also fed into the hydroformylation reactor system 3. For the sake of simplicity, the hydroformylation reactor system is shown in FIG. 1 as a single unit, but it advantageously comprises a series of sequentially-connected hydroformylation reactors.

A recycle catalyst stream 13, which comprises a transition metal-organomonophosphite ligand complex catalyst, preferably, a rhodium-organomonophosphite ligand complex catalyst, and optionally free or uncomplexed organomonophosphite ligand, solubilized and dissolved in a liquid heavy by-products phase is also fed into the hydroformylation reactor system 3, wherein hydroformylation of the olefin occurs to produce a crude hydroformylation product stream 4 comprising one or more aldehyde products, one or more heavy by-products, one or more unconverted olefinic reactants, the transition metal-organophosphite ligand complex catalyst, free organophosphite ligand, and lights including inert lights, carbon monoxide, and optionally hydrogen. In one embodiment of the invention, the crude hydroformylation product stream 4 is a stream comprising liquid and gas, which gas may be partially dissolved in the liquid. A reactor vent stream (not shown) comprising primarily light components, including inert lights, hydrogen, and carbon monoxide, can be taken overhead as a gaseous stream from the reactor system 3 from any one or more of the reactors therein.

The liquid hydroformylation product stream 4 is fed into a stripping gas vaporizer unit 6, from which an overhead gas stream 7 is obtained comprising one or more aldehyde products, one or more unconverted olefinic reactants, a portion of the heavy by-products, and lights including one or more inert lights, carbon monoxide, and optionally hydrogen. The overhead gas stream 7 from the stripping gas vaporizer is fed into a product condenser 9 from which a condenser overhead gas stream 10 is obtained comprising a portion of the one or more olefinic reactants, and a portion of the inert lights, carbon monoxide, and optionally hydrogen. From the condenser 9 a liquid product stream 8 is obtained comprising one or more aldehyde products, the portion of heavy by-products from the overhead gas stream from the vaporizer, and the balance of the unconverted olefinic reactant(s). The condenser overhead gas stream 10 is split into a recycle stream 16, which is sent back to the stripping gas vaporizer 6 via blower 11, and a stream 15 that can be recycled to the hydroformylation reactor system 3, or flared, or used as a fuel, or used in another downstream process. The recycle stream 16 comprises one or more unconverted olefin reactants and lights including one or more inert lights, carbon monoxide, and optionally hydrogen. Stream 15 comprises one or more unconverted olefin reactants and lights including one or more inert lights, carbon monoxide, and optionally hydrogen. From the stripping gas vaporizer 6, a recycle catalyst stream 13 is obtained as a vaporizer tails stream comprising the balance of the heavy by-products, the transition metal-organophosphite ligand complex catalyst, and optionally, free organophosphite ligand. Recycle catalyst stream 13 is recycled as a liquid catalyst stream back to the Oxo reactor system 3.

Stream 12 can be used to add CO directly to vaporizer 6 and/or anywhere in stream 16 prior to entry into the vaporizer 6 via stream 14. The CO partial pressure in the vaporizer can be measured directly in the vaporizer or indirectly by analyzing one or more appropriate vaporizer input and/or output streams such as, for example, an appropriate selection of streams 7, 10, 14, 15, and/or 16.

Without the addition of CO, the partial pressure of CO in the overhead gas recycle stream will vary as a function of the operating temperature of the condenser 9. In such a case, manipulation of the operating temperature of the condenser 9 provides little control over the desired quantity of CO to be recycled to the vaporizer 6 for stabilization of the hydroformylation catalyst and does not provide a sufficient amount of CO to reach the desired, e.g., greater than 5 psia (34 kPa) up to 15 psia (103 kPa), CO partial pressure. Thus, one feature of the invention is the addition of CO to the vaporizer 6, e.g., via line 14 as shown in FIG. 1. However, the addition of CO via line 12 must be balanced against increasing the total pressure of the vaporizer to avoid issues with reduced volatilization of aldehyde product thus adjustments to the 15/16 split ratio must accompany changes in the flow rate of stream 12. By adjusting this ratio, the CO partial pressure can be maintained as high as 15 psia (110 kPa) while the total pressure remains constant and sufficiently low to facilitate product and heavies removal. Even at the reduced total pressure, the use of a CO-rich stream 12 and the vent stream 15 reduces the H₂ content which improves the catalyst stability in the vaporizer without needing to change the condenser (9) temperature.

A substantial amount of the CO added via line 12 will be recycled via line 16 depending on the line 16/line 15 split ratio. The amount of H₂ will also partition similarly. This recycling reduces the total amount of flow from line 12 needed to maintain the CO partial pressure in the stripping gas vaporizer as compared to conventional vaporizers due to the relatively low solubility of CO in the liquid product outlet streams. However, while the CO partial pressure will build up under such a recycle process, so will the hydrogen content unless the ratio of H₂ to CO in line 12 is lower than the ratio of CO and H₂ coming into the process via line 4. The flow of line 12 is regulated to maintain the observed CO partial pressure in the vaporizer within the desired ranges while reducing the H₂ content. This line can also be used to introduce CO-containing stripping gas during startup where suitable gas from the upstream process may not be available. In various embodiments of the invention, streams equivalent to stream 12 may be added anywhere in the vaporizer. However, it is preferred to introduce CO to the vaporizer by mixing the make-up CO feed stream with the stripping gas 16 prior to entry into the vaporizer as stream 14.

Stream 12 advantageously is a CO-containing stream, and preferably is substantially free of sulfur- or halide-containing impurities and oxygen (O₂). The source of stream 12 may be the same source as the source of CO and H₂ to the hydroformylation reaction zone, but is preferably enriched in CO using conventional techniques such as pressure swing adsorption, membrane separation, or other known technologies. These concentration technologies may be fed with fresh syngas and/or one of the vents from the hydroformylation unit. In general, the higher the CO content in stream 12, the smaller the flow of vent stream 15 which results in lower vent losses. In other words, higher CO content in stream 12 changes the line 16/line 15 split ratio to reduce the vent flow (and thus vent losses). Conversely, should lines 4 and 12 contain substantial hydrogen, then the line 16/line 15 split ratio is adjusted lower (higher line 15 flow).

The reaction fluid from the hydroformylation reactors can be fed directly into the stripping gas vaporizer. A stripping gas vaporizer is shown in FIG. 1 as a single unit 6, but the vaporizer may comprise a series of sequentially-connected vaporizers that operate at different pressures.

As shown in FIG. 2, the reaction fluid can be fed first into a flash vessel 17 to let down pressure and remove undissolved, reactive and inert gases, after which the remaining liquid can be fed to the stripping gas vaporizer via line 4c. For example, a flash vessel, operating at a pressure in-between the reactor (3) pressure and the vaporizer (6) pressure, enables the removal of gases such as hydrogen, CO₂, methane, nitrogen, argon, and the like before they enter the vaporizer. This not only allows the concentration of these gases to be rapidly lowered, but helps prevent them from accumulating in the recycled stripping gas. Accumulation of such gases would require a higher fresh CO feed rate (stream 12) and purge flow rate (stream 15) in order to achieve the desired CO partial pressure and low H₂ partial pressure in the vaporizer. An optional cooler 18 may be present on stream 4 prior to the knockout pot 17 to reduce the aldehyde and olefin losses via stream 19 or there can be a condenser on stream 19 (not shown) to recover and recycle volatile aldehydes and olefins. Thus using a flash vessel prior to the vaporizer can extend the viable operating pressure of the vaporizer (i.e., allows for a lower total pressure) and may result in more economical operation.

The composition of the reaction fluid from the hydroformylation reactor, exclusive of the transition metal-organophosphorous ligand complex catalyst and any free ligand, advantageously comprises from about 38 to about 58 weight percent of one or more aldehyde products, from about 16 to about 36 weight percent heavies by-products, from about 2 to about 22 weight percent unconverted olefinic reactants, from about 1 to about 22 weight percent inert lights, from about 0.02 to about 0.5 weight percent carbon monoxide, and less than about 100 ppmw hydrogen, the total adding up to 100 weight percent.

The vaporizer hardware may be conventional in design, and many examples are known to the skilled person. The vaporizer is advantageously designed to include a vertical series of tubes within a heat exchanger. Optimum vaporizer dimensions (number of tubes, diameter and length) are determined by the plant capacity, and can be readily determined by one skilled in the art. Examples of vaporizers and their use are described in U.S. Pat. No. 8,404,903.

In order to maintain the CO partial pressure of the invention, it may be necessary to discharge a portion of the recycled stripping gas by means of a vent stream 15. The aldehyde, unreacted olefins and alkanes entrained in the vent stream can be recovered by condensation. The condensation can be conducted in any suitable condenser using any suitable heat transfer fluid. Examples of such fluids include, e.g., chilled water, brine or other salt solutions, DOWTHERM brand heat transfer fluid, or other heat exchange fluids, including mixtures thereof.

Since the stripping gas vaporizer and the product condenser can be operated at essentially constant pressure, no extensive compression of gaseous streams is required in some embodiments of the inventive process. A blower or fan can be suitably used for the circulation of the recycle gas from the product condenser to the stripper. Compared to a compression unit, a blower or fan involves considerably less capital expense and maintenance expense; however, a compression unit can be used if desired. Generally, the stripper and product condenser are operated at a pressure in the range of from 1.5 bar absolute (150 kPa) to 4 bar absolute (400 kPa), preferably from 2 to 3 bar absolute (200-300 kPa).

The CO partial pressure in the stripping gas vaporizer advantageously is maintained within the range of greater than 5 psia (34 kPa) to less than 15 psia (103 kPa) by adding a CO-containing stream, e.g., as shown in FIG. 1 via line 12 and adjusting the 16/15 ratio. In one embodiment of the invention, the vaporizer is operated at a temperature that is high enough to remove at least a portion of the heavies from the product fluid in the gas overhead stream, yet low enough to ensure stability of the catalyst and organophosphorous ligand in the vaporizer. Preferably, the vaporizer process outlet temperature is at least 70° C., and more preferably is at least 85° C. Preferably, the vaporizer process outlet temperature is not more than 120° C., and more preferably is not more than 110° C. The vaporizer total pressure advantageously is at least 5 psia (69 kPa), and preferably is at least 20 psia (138 kPa), and most preferably is at least 25 psia (172 kPa). The vaporizer total pressure is advantageously not more than 40 psia (276 kPa), and preferably is not more than 35 psia (241 kPa) and most preferably not more than 30 psia (207 kPa). The CO partial pressure is less than 15 psia (103 kPa), preferably less than 12 psia (83 kPa) and most preferably less than 10 psia (69 kPa) but should be above 5 psia (34 kpa). The vaporizer advantageously operates with a mass ratio of crude liquid product feed to liquid tails ranging from 1.5/1 to 5/1, preferably, from 2/1 to 3/1. The mass ratio of crude liquid product feed to recycle gas feed to the vaporizer is preferably greater than 0.1/1, more preferably greater than 0.25/1, but preferably less than 2/1, and more preferably less than 1/1. In one embodiment of the invention, in the vaporizer, the H₂ partial pressure relative to the CO partial pressure is at most 10:90, preferably 5:95, and most preferably 2:98, respectively. In a preferred embodiment, the H₂ partial pressure represents less than 1% of the total pressure in the vaporizer. In one embodiment, the invention is a process as described herein wherein the stripping gas vaporizer and the product condenser are operated essentially isobarically.

The overhead gas stream from the vaporizer is fed into a condenser. Any cooling medium desired can be employed with the condenser, and the type of cooling medium is not particularly critical. In one embodiment of the invention, the condenser employs conventional water cooling. Water is the preferred cooling medium at an operating temperature ranging from above freezing (i.e., greater than 0° C.) to about 50° C., preferably, from about 34° C. to about 45° C.

The overhead stream from the condenser advantageously is split into a gas vent stream and a gas recycle stream to the vaporizer. In one embodiment of the invention, the gas recycle stream from the condenser to the vaporizer comprises less than 5 weight percent of aldehyde products.

The use of syngas containing roughly 50 mol % H₂ for the recycle stream increases the total pressure of the vaporizer, thus purified CO is preferred. If syngas is used, it need not be at the same H₂/CO ratio as syngas fed to the hydroformylation unit, since little of this syngas will be present in stream 13 to be recycled back to the hydroformylation system. In one embodiment, the source of this CO-containing stream 12 includes a reactor vent stream that has been passed through a condenser to remove most of the condensables, such as aldehyde product and olefin starting materials, optionally in conjunction with a membrane separator or other separation device to further enrich the stream with CO.

Advantageously, the process of the invention results in lower rhodium loss and thereby lower catalyst costs compared to a comparative process that does not maintain the indicated CO partial pressure with reduced H₂ content. In one embodiment of the invention, the crude product stream is obtained by contacting CO, H₂, an olefin and a catalyst comprising rhodium and an organophosphite ligand in a reaction zone under hydroformylation reaction conditions to produce an aldehyde product in a crude product stream. In one embodiment of the invention, the process further comprises removing, as a tails stream from the vaporizer, a liquid recycle catalyst stream comprising the transition metal-organophosphite ligand complex catalyst and heavy by-products.

Illustrative non-optically active aldehyde products include e.g., propionaldehyde, n-butyraldehyde, isobutyraldehyde, n-valeraldehyde, 2-methyl 1-butyraldehyde, hexanal, hydroxyhexanal, 2-methyl 1-heptanal, nonanal, 2-methyl-1-octanal, decanal, adipaldehyde, 2-methylglutaraldehyde, 2-methyladipaldehyde, 3-hydroxypropionaldehyde, 6-hydroxyhexanal, alkenals, e.g., 2-, 3- and 4-pentenal, alkyl 5-formylvalerate, 2-methyl-1-nonanal, 2-methyl 1-decanal, 3-propyl-1-undecanal, pentadecanal, 3-propyl-1-hexadecanal, eicosanal, 2-methyl-1-tricosanal, pentacosanal, 2-methyl-1-tetracosanal, nonacosanal, 2-methyl-1-octacosanal, hentriacontanal, 2-methyl-1-triacontanal, and the like.

Illustrative optically active aldehyde products include (enantiomeric) aldehyde compounds prepared by the asymmetric hydroformylation process of this invention such as, e.g., S-2-(p-isobutylphenyl)-propionaldehyde, S-2-(6-methoxy-2-naphthyl)propionaldehyde, S-2-(3-benzoylphenyl)-propionaldehyde, S-2-(3-fluoro-4-phenyl)phenylpropionaldehyde, and S-2-(2-methylacetaldehyde)-5-benzoylthiophene.

### Specific Embodiments of the Invention

All parts and percentages in the following examples are by weight unless otherwise indicated. Pressures in the following examples are given as absolute pressure unless otherwise indicated. All manipulations such as preparation of catalyst solutions are done under inert atmosphere unless otherwise indicated. Comparative Experiments are not embodiments of the invention.

Rhodium analyses are performed by air/acetylene atomic absorption (AA) or by inductively coupled plasma (ICP). It has been found that air/acetylene AA will not reliably quantify clustered rhodium, but nonetheless, this method can still be used to indicate “rhodium loss” (e.g., the rhodium is clustered or otherwise no longer in solution and likely less active). ICP is believed to detect all rhodium in the sample regardless of form due to the high temperature of the plasma, thus a decline in rhodium as measured by ICP indicates that a portion of the rhodium is no longer in solution. Color change (starting from a colorless or light-yellow solution), darkening or formation of a black film or solids is also indicative of catalyst degradation.

Gas compositions (mole %) are measured by gas chromatography (GC) and partial pressures are then calculated based on the total pressure using Raoult's law. It should be understood that the strip gas typically includes trace components in addition to those listed (e.g. <0.5 psia).

### General Procedure A—Single Pass Gas Stripping Reactor

Unless otherwise indicated, examples and comparative experiments are conducted in 90 mL flow-through Fisher Porter reactors equipped with means for accurate control of temperatures and gas flows. Reactor off gases are analyzed by online GC to determine partial pressures. Mixing in the flow-through reactor is effected by continuous gas flow via a sparger at the bottom of the reactor. This reactor design is described in detail in U.S. Pat. No. 5,731,472, the teachings of which are incorporated by reference.

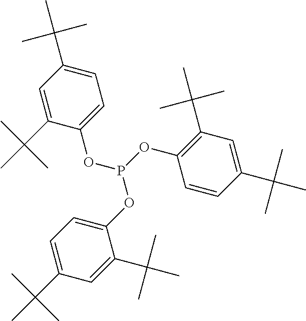 [SMILES: CC(C)(C)c1ccc(OP(Oc2ccc(C(C)(C)C)cc2C(C)(C)C)Oc2ccc(C(C)(C)C)cc2C(C)(C)C)c(C(C)(C)C)c1]

### Examples 1-4 and Comparative Example

Following the General Procedure A, Solutions of rhodium and Ligand A at various concentrations in tetraglyme are charged to individual reactors. Following an overnight at 75° C. contact with 1:1:0.1 CO:H₂:Propylene gas, the reactors are flushed with N₂ for about 60 minutes and then sealed under a set pressure with a partial pressure of 2 psi CO. After 2 and 7 days, the reactors are sampled to determine rhodium loss by AA, and the results are summarized in Table I.

TABLE I

| | [Rh] | [L] wt % | | CO | Rh | Rh | | |
|---|---|---|---|---|---|---|---|---|
| | ppm | (L/Rh mole | | PP | (ppm) | (ppm) | | % Rh |
| Exp | Target | Ratio) | | (psi) | 2 days | 7 days | Appearance | Loss |
| Comparative | 300 | 0.5 | (4) | 2 | 87.54 | 50.25 | black hazy | 83.25% |
| 1 | 77 | 0.5 | (12) | 2 | 77.13 | 55.19 | black hazy | 28.32% |
| 2 | 33 | 0.5 | (25) | 2 | 38.68 | 33.35 | clear yellow | −1.06% |
| 3 | 77 | 1 | (22) | 2 | 75.28 | 60.47 | clear yellow | 21.47% |
| 4 | 77 | 2 | (44) | 2 | 70.91 | 70.56 | clear yellow | 8.36% |

The conditions are meant to model the worst-case conditions in a vaporizer with low CO and traces of residual hydrogen and olefin. There are two generally accepted criteria for catalyst stability; appearance (black and/or hazy solutions are indicative of cluster formation) and actual metal analysis wherein the transition metal is no longer in solution. As shown in Table 1, low Ligand/Rhodium ratios (less than 20) exhibited the characteristic formation of “Rhodium black” but exhibited improved (lower) rhodium loss by AA compared to the control. Increasing the ligand:metal ratio gave improved stability by both criteria. Combining both high ligand/rhodium values and reduced overall rhodium level gave the best result.

1. A continuous hydroformylation process comprising:
(A) feeding a reaction fluid comprising one or more products, one or more heavy by-products, a transition metal-organophosphite ligand complex catalyst, one or more unconverted reactants, and one or more inert lights into a vaporizer having an average CO partial pressure;
(B) removing from the vaporizer an overhead gas stream comprising one or more products, one or more unconverted reactants, one or more inert lights, and a portion of the heavy by-products, and feeding said overhead gas stream into a condenser;
(C) removing from the condenser a condenser overhead gas stream comprising one or more unconverted reactants and one or more inert lights;
(D) recycling at least a portion of said condenser overhead gas stream to the vaporizer and/or a reaction zone;
(E) removing as a tails stream from the vaporizer, a liquid recycle catalyst stream comprising the transition metal-organophosphite ligand complex catalyst and the balance of the heavy by-products;
wherein the average CO partial pressure in the vaporizer is maintained at less than 15 psia (110 kPa).

2. The continuous hydroformylation process of claim 1 where the average CO partial pressure in the vaporizer is maintained at less than 15 psia by the following process:
a. feeding a crude product stream comprising one or more products, one or more heavy by-products, a transition metal-organophosphite ligand complex catalyst, one or more unconverted reactants, and one or more inert lights into a vaporizer;
b. removing from the vaporizer an overhead gas stream comprising one or more products, one or more unconverted reactants, one or more inert lights, hydrogen, carbon monoxide, and a portion of the heavy by-products, and feeding said overhead gas stream into a condenser;
c. removing from the condenser a condenser overhead gas stream comprising one or more unconverted reactants and one or more inert lights;
d. recycling at least a portion of said condenser overhead gas stream to the vaporizer and/or a reaction zone;
e. introducing to the vaporizer, in addition to the condenser overhead gas stream, a gas stream comprising CO, such that the average CO partial pressure in the vaporizer is less than 15 psia (103 kPa); wherein the molar ratio of CO to H₂ in the gas stream being added is higher than the ratio present at step b., and wherein the vaporizer total pressure, CO partial pressure, and H₂ partial pressure are controlled by the fraction of the condenser overhead gas stream recycled in step d.;
f. removing as a tails stream from the vaporizer, a liquid recycle catalyst stream comprising the transition metal-organophosphite ligand complex catalyst and the balance of the heavy by-products.

3. The continuous hydroformylation process of claim 1 where the average CO partial pressure in the vaporizer is maintained at less than 15 psia by the following process:
a. feeding a crude product stream comprising one or more products, one or more heavy by-products, a transition metal-organophosphite ligand complex catalyst, one or more unconverted reactants, and one or more inert lights into a depressurization zone wherein a gaseous purge is removed and the liquid phase is sent to a vaporizer; wherein the depressurization zone removes undissolved H2 and optionally inert gases such that the amount of H2 being introduced to the vaporizer is reduced to allow reduced total vaporizer pressure;
b. removing from the vaporizer an overhead gas stream comprising one or more products, one or more unconverted reactants, one or more inert lights, hydrogen, carbon monoxide, and a portion of the heavy by-products, and feeding said overhead gas stream into a condenser;
c. removing from the condenser a condenser overhead gas stream comprising one or more unconverted reactants and one or more inert lights;
d. recycling at least a portion of said condenser overhead gas stream to the vaporizer and/or a reaction zone;
e. introducing to the vaporizer, in addition to the condenser overhead gas stream, a gas stream comprising CO, such that the average CO partial pressure in the vaporizer is less than 15 psia (103 kPa); wherein the vaporizer total pressure, CO partial pressure, and H₂ partial pressure are controlled by the fraction of the condenser overhead gas stream recycled in step d.;
f. removing as a tails stream from the vaporizer, a liquid recycle catalyst stream comprising the transition metal-organophosphite ligand complex catalyst and the balance of the heavy by-products.

4. The continuous hydroformylation process of claim 1 where the average CO partial pressure in the vaporizer is maintained at less than 15 psia by the following process:
a. feeding a crude product stream comprising one or more products, one or more heavy by-products, a transition metal-organophosphite ligand complex catalyst, one or more unconverted reactants, and one or more inert lights into a vaporizer;
b. removing from the vaporizer an overhead gas stream comprising one or more products, one or more unconverted reactants, one or more inert lights, hydrogen, carbon monoxide, and a portion of the heavy by-products, and feeding said overhead gas stream into a condenser;
c. removing from the condenser a condenser overhead gas stream comprising one or more unconverted reactants and one or more inert lights;
d. recycling at least a portion of said condenser overhead gas stream to the vaporizer and/or a reaction zone;
e. introducing to the vaporizer, in addition to the condenser overhead gas stream, a gas stream comprising CO, such that the average CO partial pressure in the vaporizer is less than 15 psia (103 kPa); wherein the vaporizer total pressure, CO partial pressure, and H₂ partial pressure are controlled by the fraction of the condenser overhead gas stream recycled in step d.;
f. removing as a tails stream from the vaporizer, a liquid recycle catalyst stream comprising the transition metal-organophosphite ligand complex catalyst and the balance of the heavy by-products; wherein the ligand:rhodium ratio of the crude product stream in step (a) is above 20:1, the transition metal concentration in the tails stream in step f. is less than 100 ppm, or both.

5. The continuous hydroformylation process of claim 1 where the average CO partial pressure in the vaporizer is maintained at less than 15 psia by two or more of the processes set forth in claims 2, 3, and 4.

6. The method of claim 1 wherein the transition metal is rhodium.

7. The method of claim 1 wherein the metal concentration is 5 to 80 ppmw in step (A).

8. The method of claim 1 wherein the ligand to transition metal ratio is above 25:1.

9. The method of claim 1 wherein the ligand is a monophosphite ligand.

10. The method of claim 1 wherein the CO partial pressure in the vaporizer is less than less than 12 psia (83 kPa) and but above 5 psia (34 kpa).

11. The method of claim 1 wherein the H₂ partial pressure is in the vaporizer less than 0.2 psi (1.4 kPa).
